(19) United States
(12) Patent Application Publication
(10) Pub. No.: US 2025/0339468 A1
(43) Pub. Date: Nov. 6, 2025

(54) INVARIANT NATURAL KILLER T CELLS FOR TREATING ACUTE RESPIRATORY DISTRESS SYNDROME (ARDS)

(71) Applicant: MINK THERAPEUTICS, INC., New York, NY (US)

(72) Inventor: Marco PURBHOO, New York, NY (US)

(73) Assignee: MINK THERAPEUTICS, INC., New York, NY (US)

(21) Appl. No.: 19/219,140

(22) Filed: May 27, 2025

Related U.S. Application Data
(63) Continuation of application No. 19/127,071, filed on Jan. 1, 0001.
(60) Provisional application No. 63/423,036, filed on Nov. 6, 2022.
(60) Provisional application No. 63/503,431, filed on May 19, 2023.

(51) Int. Cl. A61K 35/17; A61P 11/00; A61P 29/00

(52) U.S. Cl. CPC A61K 35/17; A61P 11/00; A61P 29/00

(57) ABSTRACT

The present disclosure, at least in part, relates to compositions comprising invariant natural kill T (iNKT) cells (e.g., unmodified, allogeneic iNKT cells), and methods of using the compositions comprising the iNKT cells for treating a disease, or a symptom or complication of a disease (e.g., viral infection, acute respiratory distress syndrome (ARDS) secondary to a primary disease (e.g., viral infection) and/or its associated organ failure).

### RELATED APPLICATIONS

This application is a continuation of U.S. patent application Ser. No. 19/127,071, filed May 2, 2025, which is a US National Phase Application under 35 U.S.C. 371 of International Patent App. No. PCT/US2023/078861, filed Nov. 6, 2023, which claims the benefit under 35 U.S.C. 119(e) of the filing date of U.S. Provisional Patent App. No. 63/423,036, filed Nov. 6, 2022, and U.S. Provisional Patent App. No. 63/503,431, filed May 19, 2023, the entire contents of each of which are incorporated by reference herein.

### BACKGROUND

Acute respiratory distress syndrome (ARDS) is an inflammatory syndrome of the lung with a high mortality rate. Most deaths attributable to ARDS are not from respiratory failure, but rather from progressive dysfunction of other organs, also known as multisystem organ failure (MSOF). Effective treatments for reducing or preventing ARDS and MSOF subsequent to ARDS have remained elusive.

### SUMMARY

The present disclosure, at least in part, relates to compositions comprising invariant natural kill T (iNKT) cells (e.g., unmodified, allogeneic iNKT cells), and methods of using the compositions comprising the iNKT cells for treating a disease, or a symptom or complication of a disease (e.g., viral infection, acute respiratory distress syndrome (ARDS) secondary to a primary disease (e.g., viral infection) and/or its associated organ failure). In some embodiments, the compositions and methods provided herein reduces inflammation (e.g., inflammation in the lung associated with viral infection). In some embodiments, compositions and methods provided herein reduces second infection (e.g., secondary bacterial and/or fungal infection after viral infection). In some embodiments, administration of a composition to a subject (e.g., a subject having ARDS secondary to a viral infection) results in improved survival, reduced inflammatory response, reduced occurrence or severity of pneumonia, and/or reduced occurrence or severity of organ failure subsequent to ARDS.

In some aspects, the present disclosure provides a method of treating a subject having a viral infection, the method comprising administering the subject a composition comprising invariant natural killer T (iNKT) cells.

In some aspects, the present disclosure provides a method for treating a subject having acute respiratory distress syndrome (ARDS) (e.g., moderate or severe ARDS), the method comprising administering the subject a composition comprising invariant natural killer T (iNKT) cells.

In some aspects, the present disclosure provides a method for reducing organ damage or prevention of organ damage in a subject at risk thereof, the method comprising administering to the subject a composition comprising invariant natural killer T (iNKT) cells. In certain embodiments of the invention, the subject at risk for organ damage has acute respiratory syndrome (ARDs) and/or a viral infection. In certain embodiments of the invention, the subject with organ damage has acute respiratory syndrome (ARDs) and/or a viral infection.

In some aspects, the present disclosure provides a method for inducing an anti-inflammatory response in a subject having acute respiratory distress syndrome (ARDS), the method comprising administering the subject a composition comprising invariant natural killer T (iNKT) cells.

In some aspects, the present disclosure provides a method of reducing or preventing concomitant infections in a subject having acute respiratory distress syndrome (ARDS), the method comprising administering to the subject a composition comprising invariant natural killer T (iNKT) cells.

In some aspects, the present disclosure provides a method reducing or preventing concomitant infections in a subject receiving invasive mechanical ventilation or veno-venous extracorporeal membrane oxygenation (VV ECMO), the method comprising administering to the subject a composition comprising invariant natural killer T (iNKT) cells. In certain embodiments, the subject receiving invasive mechanical ventilation or VV ECMO has acute respiratory distress syndrome (ARDS).

In certain embodiments, the present disclosure provides a method for reducing or preventing a hospital acquired infection in a subject at risk thereof, the method comprising administering to the subject a composition comprising invariant natural killer T (iNKT cells). In certain embodiments, the subject at risk for acquiring a hospital infection has a viral infection. In certain embodiments, the subject at risk for acquiring a hospital infection is receiving invasive mechanical ventilation or veno-venous extracorporeal membrane oxygenation (VV ECMO). In certain embodiments of the invention, the subject at risk for acquiring a hospital infection has acute respiratory distress syndrome (ARDS).

In some embodiments, the iNKT cells are unmodified.

In some embodiments, the iNKT cells are derived from a donor that is not the subject. In some embodiments, the iNKT cells are allogeneic. In some embodiments, the iNKT cells are isolated from peripheral blood mononuclear cells and are expanded ex vivo.

In some embodiments, the donor is a human.

In some embodiments, at least 90% of the cells in the composition are iNKT cells. In some embodiments, at least 95% of the cells in the composition are iNKT cells.

In some embodiments, the ARDS is associated with a viral infection. In some embodiments, the viral infection is caused by coronavirus, influenza virus, enterovirus, rhinovirus, parainfluenza virus, adenovirus, respiratory syncytial virus (RSV), human metapneumovirus. In some embodiments, the coronavirus is severe acute respiratory syndrome coronavirus-2 (SARS-CoV-2), severe acute respiratory syndrome coronavirus (SARS-CoV-1), or Middle East Respiratory Syndrome Coronavirus (MERS-CoV). In some embodiments, the influenza virus is H1N1 influenza, H5N1 influenza, or H7N9 influenza.

In some embodiments, the ARDS is associated with sepsis, trauma (e.g., severe trauma with shock and multiple transfusions), cardiopulmonary bypass, transfusions of blood products, and severe burns.

In some embodiments, the subject does not receive mechanical ventilation

In some embodiments, the subject is on mechanical ventilation while being administered the composition.

In some embodiments, the subject is refractory to mechanical ventilation.

In some embodiments, the subject receives extracorporeal membrane oxygenation (ECMO). In some embodiments, the extracorporeal membrane oxygenation is veno-venous extracorporeal membrane oxygenation (VV ECMO). In some embodiments, there is no oxygenator failure due to clogging.

In some embodiments, the administration of the composition does not induce cytokine release syndrome.

In some embodiments, the administration of the composition improves survival of the subject relative to a subject that is not administered the composition.

In some embodiments, the administration of the composition induces an anti-inflammatory response in the subject as measured by one or more cytokines, wherein the one or more cytokines comprises: IL-1α/1β, IL-6, ferritin, C reactive protein (CRP), IL-2, IL-5, IL-7, IP-10, IL-15, IL-12p70, IFNγ, TFNα, IL-17A, IL-1RA, IL-4, IL-10, IL-13, IL-8, MCP-1, MIP-1α, VEGF, or VEGF-D.

In some embodiments, the administration of the composition reduces the occurrence of concomitant infections (e.g., VAP) relative to a subject that is not administered the composition. In some embodiments, the concomitant infections are hospital acquired infections. In some embodiments, the hospital acquired infections comprises Klebsiella aerogenes, catheter-related bloodstream infection due to Candida albicans, ventilator-associated pneumonia (VAP) due to multidrug-resistant Pseudomonas aeruginosa (MDRP).

In some embodiments, the administration of the composition reduces the occurrence of one or more organ failures relative to a subject that is not administered the composition. In some embodiments, the organ failure comprises renal failure, hepatic failure, hematologic failure, and/or neurological failure. In some embodiments, the organ failure is renal failure.

In some embodiments, the subject is administered 80×10⁶ to 2000×10⁶ iNKT cells. In some embodiments, the subject is administered 100×10⁶ iNKT cells. In some embodiments, the subject is administered 300×10⁶ iNKT cells. In some embodiments, the subject is administered 1000×10⁶ iNKT cells.

In some embodiments, the administration is via intravenous injection or intravenous infusion.

In some embodiments, the subject is administered with the composition once. In some embodiments, the subject can be repeatedly dosed one or more times after the initial dosing.

In some embodiments, the subject is also administered dexamethasone and/or remdesivir.

In some embodiments, the administration results in improved lung function of the subject compared to the lung function of the subject prior to the administration. In some embodiments, the administration results in increased lung volume of the subject compared to the lung volume of the subject prior to the administration. In some embodiments, the administration results in increased lung parenchyma stability of the subject compared to the stability of lung parenchyma of the subject prior to the administration.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 is a schematic representation showing anti-viral mechanisms by invariant Natural Killer T (iNKT) cells.

FIG. 2 shows patients' COVID-19 diagnosis date in the context of SARS-CoV-2 strain prevalence in the USA.

FIG. 3 shows the on-study 30-day survival compared to control population survival outcomes. On-study patients showed 70% survival compared to the 10% survival in the control group.

FIGS. 4A-4F show treatment with iNKT cell therapy did not induce Cytokine Release Syndrome (CRS). FIG. 4A shows IL-1α level pre- and post-iNKT cell treatment. FIG. 4B shows IL-1β level pre- and post-iNKT cell treatment. FIG. 4C shows IL-6 level pre- and post-iNKT cell treatment. FIG. 4D shows ferritin level after iNKT cell treatment. FIG. 4E shows C-reactive protein (CRP) level after iNKT cell treatment. FIG. 4F shows D-Dimer level after iNKT cell treatment.

FIG. 5 shows the 90-day survival curve of patients undergoing vein-to-vein extracorporeal membrane oxygenation (VV ECMO) with iNKT cell treatments compared to control group (patients undergoing VV ECMO but not receiving iNKT cell treatment). The VV ECMO+iNKT cell therapy group showed 75% survival whereas the control group showed only 30% survival.

FIGS. 6A-6D show peripheral persistence of iNKT cells in blood of patients under invasive mechanical ventilation (IMV) or VV ECMO. FIGS. 6A-6C show cohort level peripheral persistence of iNKT cell in patient PBMCs by digital PCR based on genetic markers unique to donor material. Each cohort a received different dose of iNKT cells. Each line represents data from one patient. Donor iNKT cells were detected up to day 6 post infusion, with likely low-level persistence detectable at the highest dose level for longer, up to the last day of sampling at day 28. Peak levels of iNKT cells demonstrate a dose-proportional relationship. Data from patients on ECMO are in red. FIGS. 6A-6B are representative results from patients under IMV who received iNKT cells. FIG. 6C includes representative results from patients under IMV or VV ECMO that received iNKT cells. FIG. 6D shows dynamics of tissue distribution of iNKT cells in a murine xenograft model demonstrating rapid translocation of iNKT cells to tissue following intravenous (i.v.) injection. The observed transient post-infusion persistence of iNKT cells in patient blood is consistent with the dynamics of blood-to-tissue distribution of iNKT cells in vivo.

FIG. 7A-7R are representative graphs showing serum cytokine levels of selected biomarkers spanning the immuno-regulatory spectrum. FIGS. 7A-7I are representative graphs showing production of pro-inflammatory cytokines post iNKT cell infusion. FIGS. 7L-7M show production of anti-inflammatory cytokines post iNKT cell infusion. FIGS. 7N-7P show production of chemotactic factor post iNKT cell infusion. FIGS. 7Q-7R show production of growth factors post iNKT cell infusion.

FIGS. 8A-8D are representative graphs showing the presence of donor specific allo-antibodies (DSA), which was determined on day of dosing and day 14. FIGS. 8A-8B show induction of DSA in patients with HLA class I matching (FIG. 8A) or HLA class II matching (FIG. 8B). FIG. 8C is a representative graph showing serum levels of DSA post-dosing, which was reduced with increased degree of HLA matching. DSA levels of combined MFI<1,000 were considered negative and not reported. FIG. 8D is a representative graph showing DSA levels measured at the time of discharge of four patients (day of discharge of patients, from left to right: 60, 28, 21, 28). DSA levels post infusion of iNKT cells peaked at day 14 and appeared to decrease afterwards (data normalized to peak DSA levels for each patient).

FIGS. 9A-9C are representative graphs showing survival, iNKT cell persistence, and anti-inflammatory cytokine production in patients on Veno-Venous Extracorporeal Membrane Oxygenation (VV-ECMO) and receiving iNKT cell therapy. FIG. 9A shows that survival was 100% at 14 days and 75% at 30 and 90 days with an average ECMO run time of 133.5 days. FIG. 9B shows that persistence of circulating iNKT cells in the VV-ECMO cohort was comparable to study patients not on ECMO. FIG. 9C shows that significantly increased levels of the anti-inflammatory cytokine IL1-RA was observed.

FIGS. 10A-10B are Chest X-Rays images showing improved lung function within 24 hours after iNKT cell infusion. FIG. 10A is the chest X-ray image pre-infusion. FIG. 10B is the chest X-ray image post-infusion.

### DETAILED DESCRIPTION

The present disclosure, at least in part, relates to compositions comprising invariant natural kill T (iNKT) cells (e.g., unmodified, allogeneic iNKT cells), and methods of using the compositions comprising the iNKT cells for treating a disease, or a symptom or complication of a disease (e.g., acute respiratory distress syndrome (ARDS) secondary to a primary disease (e.g., viral infection)). In some embodiments, the present disclosure is based on the unexpected observation that administration of iNKT cell to a subject (e.g., a subject having ARDS secondary to a viral infection) results in improved survival (e.g., 70% 30-day survival in subjects on invasive mechanical ventilation (IMV) who received iNKT cell therapy relative to 10% 30-Day survival in subjects on IMV but did not receive iNKT cell therapy; and 75% 90-day survival in subjects on Veno-Venous Extracorporeal Membrane Oxygenation (VV ECMO) who received iNKT cell therapy relative to ˜30% 90-Day survival in subjects on VV ECMO but did not receive iNKT cell therapy), reduced inflammatory response, reduced occurrence or severity of concomitant infections and pneumonia in subjects receiving 1000×10⁶ iNKT cells, and/or reduced occurrence or severity of organ failure subsequent to ARDS. Further, iNKT cell therapy provides at least the following benefits: (i) demonstrates a favorable safety profile (e.g., no neurotoxicity or grade≥3 TRAE were observed); (ii) demonstrates transient persistence in the periphery consistent with in vivo data describing rapid translocation of iNKT cells from blood into tissues; (iii) opportunity for repeated dosing (while alloantibodies were detected after iNKT cell administration and correlates with degree of HLA matching, the antibody response is transient); and (iv) ability to treat viral diseases and infections (a reduced incidence of Pneumonia was seen in patients treated at the highest dose of iNKT cell therapy).

In addition, the present disclosure provides a variant agnostic approach for ARDS (e.g., COVID-19 ARDS) and is the first immune cell therapy used in patients on ECMO. Despite trends toward improved mortality in severe COVID-19 respiratory failure, largely due to early corticosteroid and anti-viral therapy, death rates have remained high (47.9% to 84.4%). Addition of Veno-Venous Extracorporeal Membrane Oxygenation (VV-ECMO) support has improved survival in ARDS patients (e.g., ARDS in COVID-19 patients), but overall mortality at 90 days is still low (i.e., approximately 47%). Complications of VV-ECMO therapy include bleeding, oxygenator failure, and hospital acquired infections, including but not limited to Klebsiella aerogenes, catheter-related bloodstream infection due to Candida albicans, ventilator-associated pneumonia (VAP) due to multidrug-resistant Pseudomonas aeruginosa (MDRP)). There is a need for multi-modal therapies in patients with severe COVID-19 respiratory failure that can augment current interventions. The present disclosure reports on the first safe administration of an allogeneic human unmodified invariant natural killer T (iNKT) cell infusion in patients with severe COVID-19 respiratory failure receiving VV-ECMO support. In contrast to previous mesenchymal stem cell therapy in ARDS patients on ECMO, no cell-therapy-associated oxygenator failure due to clogging of filters was observed.

The foregoing and other aspects, implementations, acts, functionalities, features and embodiments of the present teachings can be more fully understood from the following description in conjunction with the accompanying drawings.

I. Therapeutic Treatments Using Invariant Natural Killer T (iNKT) Cells

In some aspects, the present disclosure provides a method of treating a subjecting having a viral infection, the method comprising administering the subject a composition comprising invariant natural killer T (iNKT) cells. As used herein, the terms “administering” or “administration” means to provide a therapeutic agent (e.g., iNKT cells) or a composition thereof (e.g., a composition comprising iNKT cells) to a subject in a manner that is physiologically and/or pharmacologically useful (e.g., to treat a disease or a symptom or complication associated with the disease in the subject). As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having acute lung injury (ALI)/acute respiratory distress syndrome (ARDS). As used herein, the term “treating” or “treatment” refers to the application or administration of a composition including one or more active agents (e.g., unmodified, allogeneic iNKT cells) to a subject, who has a target disease or disorder (e.g., ARDS), a symptom or complication of the disease/disorder (e.g., respiratory distress, multiple organ failure), or a predisposition or primary indication toward the disease/disorder (e.g., viral infection), with the purpose to cure, heal, alleviate, relieve, alter, remedy, ameliorate, improve, or affect the disorder (e.g., ARDS), the symptom or complication of the disease (e.g., respiratory distress, multiple organ failure), or the predisposition or primary indication toward the disease or disorder (e.g., viral infection). Alleviating a target disease/disorder includes delaying or preventing the development or progression of the disease, reducing disease severity, and/or promoting survival.

iNKT cell therapy elicits anti-viral effects in at least the following aspects: (i) recognition of CD1d ligands in diseased tissue and activation through the invariant TCR; (ii) recognition of stress-signals through activating NK receptors (e.g., NKG2D, DNAM1); (iii) modulation and/or destruction of myeloid suppressor cells and inflammatory monocytes: (iv) recruitment and activation of NK and T cells through cytokine secretion; (v) reversal of T cell exhaustion; and (vi) cytokine mediated control of bacterial infections, including pneumonia. Further, iNKT cells are substantially devoid of alloreactivity, being restricted for the monomorphic CD1d molecule, allowing their possible use in an “off the shelf” and in a donor-unrestricted manner (e.g., without causing graft-versus-host disease (GvHD)).

In some aspects, the present disclosure also provides a method for treating a subject having acute respiratory distress syndrome (ARDS) (e.g., moderate or severe ARDS), the method comprising administering the subject a composition comprising invariant natural killer T (iNKT) cells. In some embodiments, the present disclosure also provides a method for reducing or preventing organ failure in a subject having acute respiratory distress syndrome (ARDS), the method comprising administering the subject a composition comprising invariant natural killer T (iNKT) cells. Acute respiratory distress syndrome (ARDS), and its milder form acute lung injury (ALI), are a spectrum of lung diseases characterized by a severe inflammatory process causing diffuse alveolar damage and resulting in a variable degree of ventilation perfusion mismatch, severe hypoxemia, and poor lung compliance (Ware et al., The acute respiratory distress syndrome. N Engl J Med 2000; 342:1334-49). ARDS is described as a rapid onset of tachypnoea and hypoxaemia, with loss of lung compliance and bilateral infiltrates on chest radiograph, in otherwise healthy young individuals. Although the ARDS precipitating illnesses differed between patients, they had similar clinical and pathological features. Clinical syndromes associated with ARDS include but are not limited to: (i) Direct lung injury such as pulmonary infection (e.g., viral or bacterial infection), pneumonia, aspiration of gastric contents, fat emboli, near drowning, inhalational injury, reperfusion pulmonary edema after transplantation, and pulmonary embolectomy; and (ii) Indirect lung injury such as sepsis, trauma (e.g., severe trauma with shock and multiple transfusions), cardiopulmonary bypass, transfusions of blood products, and severe burns. In some embodiments, the subject has ARDS secondary to a viral infection including but not limited to coronavirus (e.g., severe acute respiratory syndrome (SARS), SARS-CoV-2, Middle East Respiratory Syndrome Coronavirus (MERS-CoV)), influenza (e.g., H1N1, H5N1, H7N9), rhinovirus, Herpes simplex virus (HSV), Cytomegalovirus, parainfluenza virus, adenovirus, respiratory syncytial virus (RSV), or human metapneumovirus.

The Berlin Definition defines ARDS patients in 3 mutually exclusive categories of ARDS based on degree of hypoxemia: mild (200 mm Hg &lt; PaO2/FIO2≤300 mm Hg), moderate (100 mm Hg &lt; PaO2/FIO2≤200 mm Hg), and severe (PaO2/FIO2≤100 mm Hg). Using the Berlin Definition, stages of mild, moderate, and severe ARDS were associated with increased mortality (ARDS Definition Task Force et al., Acute Respiratory Distress Syndrome: the Berlin Definition, JAMA. 2012 Jun. 20; 307(23):2526-33).

In some embodiments, patients with ARDS are often mechanically ventilated during the course of their illness. Invasive mechanical ventilation (IMV) requires the patient be intubated with an endotracheal tube (ETT) and a mechanical ventilator (as opposed to noninvasive ventilation in which the interface is a face mask). IMV helps stabilize patients with hypoxemic and hypercapnic respiratory failure, decreases inspiratory work of breathing, redistributes blood flow from exercising respiratory muscles to other tissues, and allows for the implementation of lung-protective (low tidal volume) ventilation. However, while IMV is an important care for patients in need (e.g., ARDS patients), mechanical ventilation itself may cause and further aggravate the lung injury, and the mortality rate in patients on IMV is still high (e.g., ˜46%).

In some embodiments, a subject has ARDS secondary to SARS-CoV-2 infection. As used herein, “SARS-CoV-2” refers to the SARS-CoV having the nucleotide sequence of GenBank: MN996527.1 (“Severe acute respiratory syndrome coronavirus 2 isolate WIV02, complete genome”), reported in Zhou et al., Nature (2020) 579: 270-273, and encompasses variants thereof having a nucleotide sequence with at least 85% sequence identity (e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater sequence identity) to the nucleotide sequence of GenBank: MN996527.1. Variants of SARS-CoV-2 of particular interest include: (i) the variant designated VUI-202012/01, which belongs to the B.1.1.7 lineage, having the canonical nucleotide sequence of GISAID accession EPI_ISL_601443; (ii) the variant designated 501Y.V2/B.1.351, having the canonical nucleotide sequence of GISAID accession EPI_ISL_768642; (iii) the variant known as B.1.1.248/P.1, having the canonical nucleotide sequence of GISAID accession EPI_ISL_792680; (iv) the variant known as B.1.617.1, having the canonical nucleotide sequence of GISAID accession EPI_ISL_2621960; and (v) the variant known as B.1.617.2, having the canonical nucleotide sequence of GISAID accession EPI_ISL_1663476. Variants of SARS-CoV-2 of particular interest also include the variants known as alpha, beta, gamma, delta, delta+, kappa, lambda, mu and omicron. The present disclosure concerns severe acute respiratory syndrome-related coronavirus (SARSr-CoV). The virology of SARSr-CoV and epidemiology of disease associated with SARSr-CoV infection is reviewed, for example, in Cheng et al., Clin Microbiol Rev (2007) 20(4): 660-694 and de Wit et al., Nat Rev Microbiol (2016) 14: 523-534.

In some embodiments, a subject having moderate ARDS (e.g., ARDS secondary to SARS-CoV-2 and/or influenza infection) as defined by the Berlin definition receives the iNKT cell therapy described herein. In some embodiments, a subject having severe ARDS (e.g., ARDS secondary to SARS-CoV-2 and/or influenza infection) as defined by the Berlin definition receives the iNKT cell therapy described herein. In some embodiments, a subject having ARDS (e.g., moderate or severe ARDS secondary to SARS-CoV-2 and/or influenza infection) is on IMV while receiving the iNKT cell therapy. In some embodiments, iNKT cell therapy improves survival in patients having ARDS (e.g., moderate or severe ARDS secondary to SARS-CoV-2 and/or influenza infection) relative to patients not receiving the iNKT cell therapy. In some embodiments, iNKT cell therapy improves survival in patients having ARDS (e.g., moderate or severe ARDS secondary to SARS-CoV-2 and/or influenza infection) and is placed on IMV relative to patients on IMV but not receiving iNKT cell therapy. In some embodiments, iNKT cell therapy achieves at least 50%, at least 55%, at least 60%, at least 65%, or at least 70% 30-day survival rate (i.e., 30-day from onset of IMV) in ARDS patients (e.g., patients having moderate or severe ARDS secondary to SARS-CoV-2 and/or influenza infection) on IMV as compared to ARDS patients (e.g., patients having moderate or severe ARDS secondary to SARS-CoV-2 and/or influenza infection) on IMV but not receiving the iNKT cell therapy.

In some patients with severe refractory ARDS (e.g., ARDS secondary to SARS-CoV-2 and/or influenza infection) to conventional therapy (e.g., when IMV cannot maintain adequate oxygenation, and/or when IMV exacerbates lung injury), extracorporeal membrane oxygenation (ECMO) is employed. In some embodiments, ARDS patients receive ECMO without previously receiving IMV. ECMO is a form of mechanical assist therapy that employs an extracorporeal blood circuit including an oxygenator and a pump. To perform standard respiratory ECMO, two vascular accesses are established, one for removal of venous blood and the other for infusion of oxygenated blood. Blood is drained from a major vein and pumped through a circuit that includes an oxygenator, which oxygenates the blood and removes carbon dioxide (CO₂), after which the oxygenated blood is returned via the other cannula. When blood is returned to the venous side of the circulation, the procedure is known as veno-venous ECMO (VV ECMO), which provides gas exchange but cannot give cardiac support. In some embodiments, an ARDS patient (e.g., a patient having ARDS secondary to SARS-CoV-2 and/or influenza infection) is on VV ECMO when receiving the iNKT cell therapy described herein (e.g., a composition comprising iNKT cells). In some embodiments, VA ECMO is selected for an ARDS patient due to the need for cardiac support associated with pulmonary hypertension, cardiac dysfunction associated with sepsis, or arrhythmia.

In some embodiments, the present disclosure provides a method of treating a subject having ARDS (e.g., ARDS secondary to SARS-CoV-2 and/or influenza infection) and is on ECMO (e.g., VV ECMO) using the iNKT cell therapy described herein. In some embodiments, iNKT cell therapy improves survival of a patient having ARDS (e.g., ARDS secondary to SARS-CoV-2 and/or influenza infection) and is on ECMO (e.g., VV ECMO), for instance, as compared to a patient receiving only ECMO treatment. In some embodiments, iNKT cell therapy achieves at least 50%, at least 55%, at least 60%, at least 65%, at least 70%, at least 75%, or at least 80% 90-day survival rate (i.e., 90-day from onset of ECMO) in ARDS patients (e.g., patients having moderate or severe ARDS secondary to SARS-CoV-2 and/or influenza infection) on ECMO as compared to ARDS patients (e.g., patients having moderate or severe ARDS secondary to SARS-CoV-2 and/or influenza infection) on ECMO but not receiving the iNKT cell therapy. In patients treated with iNKT cell therapy, no cell-therapy-associated oxygenator failure due to clogging of filters is observed as is typically seen with mesenchymal stem cell therapy in ARDS patients on ECMO.

Due to the proinflammatory aspects of iNKT cells, cytokine release syndrome (CRS) associated with cell therapy (e.g., CAR T therapy), and previous reports regarding activation of iNKT cells exacerbating acute lung injury (see, e.g., Aoyagi, et al., Activation of pulmonary invariant NKT cells leads to exacerbation of acute lung injury caused by LPS through local production of IFN-γ and TNF-α by Gr-1+ monocytes, International Immunology, Volume 23, Issue 2, February 2011, Pages 97-108), the present disclosure also in part relates to the unexpected observation that iNKT cell therapy does not induce cytokine release syndrome (CRS), but promotes an anti-inflammatory response in ARDS patients (e.g., patients having ARDS secondary to SARS-CoV-2 and/or influenza infection). Cytokine release syndrome (CRS) is a systemic inflammatory response with outpouring of the pro-inflammatory cytokines due to a stimulus triggered by a variety of factors such as infections, immunotherapy, especially those involving cell therapy, immune cell engagers (e.g., T cell engager), and antibody-based therapies. Further, severe respiratory infection (e.g., SARS-CoV-2 or influenza infection) is often associated with rapid virus replication, massive inflammatory cell infiltration and elevated pro-inflammatory cytokine/chemokine responses resulting in acute lung injury (ALI), and acute respiratory distress syndrome (ARDS). Recent studies in experimentally infected animal suggest a role for virus-induced immunopathological events in causing fatal pneumonia after infections. Cytokines are signaling molecules that can mediate and regulate the human body's immune response and inflammation, which is protective in nature under normal circumstances. However, when the levels of cytokine are too high, which overly stimulates the immune response, healthy cells are destroyed and essential organs are damaged. CRS can be present with a variety of symptoms ranging from flu-like symptoms to severe multi-organ system failure or even death. In some embodiments, subjects having ARDS (e.g., ARDS secondary to SARS-CoV-2 and/or influenza infection) receiving iNKT cell therapy are monitored for the onset of CRS as measured by production of pro-inflammatory cytokines. In some embodiments, pro-inflammatory cytokines involved in CRS include but are not limited to IFN-γ, IL-1α/1β, IL-5, IL-6, IL-7, IL-12, IL-17A, IP-10, TGFβ, CCL2, CCL5, CCL7, CXCL10, CXCL9, IL-8, ferritin, C-reactive protein (CRP), D-Dimer, TNF-α, MCP01, or MIP-1α. Unexpectedly, iNKT cell therapy does not induce CRS in ARDS patients (e.g., patients having ARDS associated with SARS-CoV-2 and/or influenza infection). In some embodiments, IL-1α/β is not detected, or within normal range as a healthy subject in ARDS patients received iNKT cell therapy. In some embodiments, ferritin, CRP and/or D-Dimer do not increase after iNKT cell therapy. In some embodiments, the iNKT cell therapy promotes an anti-inflammatory response in ARDS patients (e.g., patients having ARDS associated with SARS-CoV-2 and/or influenza infection). In some embodiments, after administration of iNKT cell therapy, an increase serum level of anti-inflammatory cytokine IL-1RA (which counteract IL-1 mediated cytokine release) in ARDS patients indicates that the iNKT cell therapy promotes an anti-inflammatory response.

Mortality in ARDS is often driven by multiple-organ system failure (see, e.g., Siuba et al., Nonpulmonary Organ Failure in ARDS: What Can We Modify?Respiratory Care May 2019, 64 (5) 610-611; Montgomery et al. Causes of mortality in patients with the adult respiratory distress syndrome. Am Rev Respir Dis 1985; 132(3):485-489; Stapleton et al., Causes and timing of death in patients with ARDS. Chest 2005; 128(2):525-532). This deterioration is thought to be secondary to extrapulmonary organ involvement due to a complex interplay between inflammatory mediators (e.g., CRS) and ongoing injury due to ventilator mechanics. A common pro-inflammatory pathway due to an initial insult (e.g., viral infection, sepsis, aspiration pneumonitis, trauma) likely exists between multiple-organ system failure and ARDS (see, e.g., Han, The acute respiratory distress syndrome: from mechanism to translation. J Immunol 2015; 194(3):855-860). Ventilator-associated lung injury has also been postulated as a precipitant for nonpulmonary organ failure (Slutsky A S et al., Multiple system organ failure. Is mechanical ventilation a contributing factor? Am J Respir Crit Care Med 1998; 157(6 Pt 1):1721-1725; Tremblay et al., Ventilator-induced injury: from barotrauma to biotrauma. Proc Assoc Am Physicians 1998; 110(6):482-488). In addition to respiratory failure, ARDS patients may develop dysfunction or failure of other organ systems including but not limited to renal failure, hepatic failure, cardiac failure, hematologic failure, and/or neurological failure. In some embodiments, the present disclosure provides a method for reducing or preventing organ failure in a subject having acute respiratory distress syndrome (ARDS), the method comprising administering the subject a composition comprising invariant natural killer T (iNKT) cells. In some embodiments, iNKT cell therapy is effective in preventing the occurrence of one or more organ failures or reducing the severity of one or more organ failures in ARDS patients received iNKT cell therapy relative to ARDS patients not receiving iNKT cell therapy. In some embodiments, iNKT cell therapy is effective in preventing the occurrence of renal failure or reducing the severity of renal failure in ARDS patients receiving iNKT cell therapy relative to ARDS patients not receiving iNKT cell therapy. In some embodiments, iNKT cell therapy is effective in reducing the number of ARDS patients having pulmonary organ failure relative to ARDS patients not receiving iNKT cell therapy.

ARDS patients are prone to develop concomitant infections (e.g., secondary pulmonary infection, namely ventilator-associated pneumonia (VAP) or infections of other organs). High frequency occurrence of VAP may be explained by traditional factors such as bronchial contamination due to endotracheal intubation and mechanical ventilation (MV) duration, but also because of impaired local (alveolar) and systemic defenses, and other specific and non-specific factors (Papazian et al., Ventilator-associated pneumonia in adults: a narrative review. Intensive Care Med. 2020; Luty et al., Pulmonary infections complicating ARDS, Intensive Care Med. 2020; 46(12): 2168-2183). Incidence of concomitant infections include but are not limited to: pneumonia, bacteremia, urinary tract infection, fungaemia, Cytomegalovirus viraemia, Lung abscess, Pneumonia klebsiella, sepsis and septic shock, or upper respiratory tract infection. Addition of Veno-Venous Extracorporeal Membrane Oxygenation (VV-ECMO) support has improved survival in ARDS patients (e.g., ARDS in COVID-19 patients), but overall mortality at 90 days is still low (i.e., approximately 47%). Complications of VV-ECMO therapy include bleeding, oxygenator failure, and hospital acquired infections, including but not limited to Klebsiella aerogenes, catheter-related bloodstream infection due to Candida albicans, ventilator-associated pneumonia (VAP) due to multidrug-resistant Pseudomonas aeruginosa (MDRP)) (see e.g., Rivosecchi et al., Secondary Infections in Patients Requiring Extracorporeal Membrane Oxygenation (ECMO) for Severe Acute Respiratory Distress Syndrome (ARDS) due to COVID-19 Pneumonia (PNA), Open Forum Infectious Diseases, Volume 8, Issue Supplement_1, November 2021, Page S260; Sun et al., Infections occurring during extracorporeal membrane oxygenation use in adult patients, The Journal of Thoracic and Cardiovascular Surgery, Volume 140, Issue 5, November 2010, Pages 1125-1132.e2). In some aspects, the present disclosure provides a method of reducing or preventing concomitant infections in a subject having acute respiratory distress syndrome (ARDS), the method comprising administering to the subject a composition comprising invariant natural killer T (iNKT) cells. In some aspects, the present disclosure also provides a method of reducing or preventing concomitant infections in a subject having acute respiratory distress syndrome (ARDS) receiving invasive mechanical ventilation or veno-venous extracorporeal membrane oxygenation (VV ECMO), the method comprising administering to the subject a composition comprising invariant natural killer T (iNKT) cells. In some embodiments, the concomitant infections in ARDS patients are hospital acquired infections, including but not limited to: Klebsiella aerogenes, catheter-related bloodstream infection due to Candida albicans, ventilator-associated pneumonia (VAP) due to multidrug-resistant Pseudomonas aeruginosa (MDRP)). In some embodiments, hospital acquired infections in ARDS patients causes pneumonia, bacteremia, urinary tract infection, fungaemia, viraemia (e.g., Cytomegalovirus viraemia), lung abscess, Pneumonia klebsiella, sepsis and septic shock, or upper respiratory tract infection.

In some embodiments, ARDS patients (e.g., patients having ARDS secondary to SARS-CoV-2 and/or influenza infection) receiving iNKT cell therapy are monitored for occurrence of concomitant infections. In some embodiments, iNKT cell therapy reduces the occurrence of concomitant infections (e.g., hospital acquired infections described herein). In some embodiments, iNKT cell therapy prevents the occurrence of concomitant infections (e.g., hospital acquired infections described herein). In some embodiments, iNKT cell therapy at a higher dose (e.g., dosage of at least 500 million cells, at least 600 million cells, at least 700 million cells, at least 800 million cells, at least 900 million cells, at least 1000 million cells, or more) is more effective in preventing concomitant infections (e.g., hospital acquired infections described herein) as compared to iNKT cell therapy at a lower dose (e.g., dosage of less than 500 million cells).

In some embodiments, ARDS patients (e.g., patients having ARDS secondary to SARS-CoV-2 and/or influenza infection) receiving iNKT cell therapy are monitored for lung function after iNKT cell administration. In some embodiments, administration of iNKT cells result in improved lung function (e.g., improved lung function within 2 hours, within s hours, within 8 hours, within 12 hours, within 16 hours, within 24 hours, within 2 days, within 3 days, within 4 days, within 5 days, within 6 days, within one week, within two week, etc.) of the subject relative to the lung function of the subject prior to administration. Lung function can be measured by appropriate lab test, e.g., lung volume test, spirometry, X-ray, CT scans, etc. For example, in some embodiments, administration of iNKT cells result in increased lung volume (e.g., increased lung volume within 2 hours, within s hours, within 8 hours, within 12 hours, within 16 hours, within 24 hours, within 2 days, within 3 days, within 4 days, within 5 days, within 6 days, within one week, within two week, etc.) of the subject relative to the lung volume of the subject prior to administration. In some embodiments, administration of iNKT cells result in increased lung parenchymal stability (e.g., increased lung parenchymal stability within 2 hours, within s hours, within 8 hours, within 12 hours, within 16 hours, within 24 hours, within 2 days, within 3 days, within 4 days, within 5 days, within 6 days, within one week, within two week, etc.) of the subject relative to the lung parenchymal stability of the subject prior to administration.

Antibodies against foreign HLA can be pathogenic in several clinical contexts, most notably in transplantation (e.g., allogeneic cell therapy), HLA antibodies can cause graft rejection. For example, mesenchymal stem cells (MSCs) therapy may provoke donors' humoral and cellular immune responses, especially in allogeneic transplants. Detection of donor specific antibodies (DSA) in the serum of transplant recipients provides clear evidence of alloantigen recognition by B cells. The generation of DSA is likely the results of indirect recognition of donor HLA presented by patient antigen presenting cells (APC) to CD4+ T cells. As a result, induction of allo-specific T CD4+ cells will activate HLA-specific IgG-producing B cells (Barrachina et al., Allo-antibody production after intraarticular administration of mesenchymal stem cells (MSCs) in an equine osteoarthritis model: effect of repeated administration, MSC inflammatory stimulation, and equine leukocyte antigen (ELA) compatibility, Stem Cell Research & Therapy volume 11, Article number: 52 (2020)). Other reports have shown that HLA antibodies are stable, even following sustained CD19+B cell depletion (e.g., Zhang et al., Stable HLA antibodies following sustained CD19+ cell depletion implicate a long-lived plasma cell source, Blood Adv (2020) 4 (18): 4292-4295). The long-lasting existence of DSA negatively impacts the use of cell therapy. iNKT cell therapy may induce the production of DSA. In some embodiments, HLA matching reduces the DSA induced by the allogeneic iNKT cell therapy described herein. In some embodiments, incidence of DSA development by the iNKT cell therapy is reduced with increased HLA class I matching. In some embodiments, contrary to the previous report, the DSA induced by the iNKT cell therapy described herein is transient, thereby enabling redosing the subject with the same iNKT cell therapy.

iNKT cell therapy (e.g., the composition comprising unmodified allogeneic iNKT cells) of the present disclosure may be administered in a manner appropriate to the disease (e.g., ARDS and its associated complications secondary to SARS-CoV-2 and/or influenza infection) to be treated or prevented. The quantity and frequency of administration will be determined by such factors as the condition of the patient, and the type and severity of the patient's disease, although appropriate dosages may be determined by clinical trials. In some embodiments, compositions of the present disclosure are formulated for intravenous administration (e.g., intravenous injection or intravenous infusion).

When “an effective amount”, or “therapeutic amount” is indicated, the precise amount of the compositions of the present disclosure to be administered can be determined by a physician with consideration of individual differences in age, weight, severity of ARDS, and condition of the patient (subject). It can generally be stated that a pharmaceutical composition comprising the iNKT cells described herein may be administered at a dosage of 104 to 10¹ cells/kg body weight, 10¹ to 10⁶ cells/kg body weight, including all integer values within those ranges. In some embodiments, iNKT cells may be administered at a dosage of 80 million (i.e., 80×10⁶) to 2000 million (i.e., 2000×10⁶) cells (e.g., at least 80 million cells, at least 90 million cells, at least 100 million cells, at least 200 million cells, at least 300 million cells, at least 400 million cells, at least 500 million cells, at least 600 million cells, at least 700 million cells, at least 800 million cells, at least 900 million cells, at least 1000 million cells, at least 1100 million cells, at least 1200 million cells, at least 1300 million cells, at least 1400 million cells, at least 1500 million cells, at least 1600 million cells, at least 1700 million cells, at least 1800 million cells, at least 1900 million cells, at least 2000 million cells, or more). iNKT cell compositions may also be administered multiple times at these dosages. The cells can be administered by using infusion techniques that are commonly known in immunotherapy (see, e.g., Rosenberg et al., New Eng. J. of Med. 319: 1676, 1988).

The administration of the subject compositions may be carried out in any convenient manner, including infusion, injection, inhalation, ingestion, intratracheal injection, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the iNKT cell compositions of the present disclosure are administered to a patient by intradermal or subcutaneous injection. In some embodiments, the iNKT cell compositions of the present disclosure are preferably administered by i.v. injection or i.v. infusion. The compositions of iNKT cells may also be injected directly into site of disease (e.g., intratracheal administration in ARDS patients on IMV).

II. Unmodified, Allogeneic Invariant Natural Killer T (iNKT) Cells

In some aspects, the present disclosure provides a composition comprising invariant natural killer T (iNKT) cells. The term “invariant Natural Killer T cells”, or “invariant NKT cells”, “iNKT cells”, or “Type I NKT cell), as used herein, refer to a population of T lymphocytes expressing a conserved semi-invariant TCR specific for lipid antigens restricted for the monomorphic MHC class I-related molecule CD1d. Natural killer T cells (NKT cells) were originally characterized in mice as T cells that express both a TCR and NK1.1 (NKR-P1a-c or CD161), a C-type lectin NK receptor. Invariant NKT (iNKT) cells express a semi-invariant αβ TCR (e.g., formed by an invariant TRAV11-TRAJ18 (4) rearrangement in mice, or the homologous invariant TRAV10-TRAJ18 chain in humans), paired with a limited set of diverse Vβ chains, predominantly TRBV1, TRBV29, or TRBV13 in mice (6) and TRBV25 in humans (see e.g., Dellabona et al., An invariant V alpha 24-J alpha Q/V beta 11 T cell receptor is expressed in all individuals by clonally expanded CD4-8− T cells. J Exp Med. (1994) 180:1171-6. 10.1084). The semi-invariant TCR recognizes exogenous and endogenous lipid antigens presented by the monomorphic MHC class I-related molecule CD1d (see e.g., Brennan et al., Invariant natural killer T cells: an innate activation scheme linked to diverse effector functions. Nat Rev Immunol. (2013) 13:101-17. 10.1038). Exogenous lipid antigens include the prototypical α-Galactosylceramide (α-GalCer) (Kawano et al., CD1d-restricted and TCR-mediated activation of valpha14 NKT cells by glycosylceramides. Science. (1997) 278:1626-9. 10.1126) and a number of bacterial-derived Ags, which can activate iNKT cells.

iNKT cells undergo a distinct developmental pathway compared to T cells, leading to the acquisition of innate effector functions already in the thymus. Thymic iNKT cells indeed express markers usually upregulated by peripheral effector/memory T cells, such as CD44 and CD69, together with distinctive NK differentiation markers, such as NK1.1 (in some mouse genetic backgrounds, CD161 in humans), CD122 (the IL-2R/IL-15R β-chain), CD94/NKG2 and Ly49(A-J), and a broad spectrum of TH1/2/17 effector cytokines. Once migrated in the periphery, iNKT cells form a tissue resident population that survey the cellular integrity and rapidly respond to local damage and inflammation, jump starting the reaction by cells of the innate and adaptive immune response.

Because iNKT cells can rapidly produce IFNγ, IL-4, or both, they have been found to play a role in various diseases by establishing a context-dependent Th1- or Th2-based immune response. In bacterial and viral infections, iNKT cells typically help in early control of the pathogen by establishing a productive Th1 response. In both mouse and human studies, roles for iNKT cells have been described in diseases associated with excessive Th1 responses like type 1 diabetes and chronic obstructive pulmonary disease. Roles have also been described for iNKT cells helping to suppress Th1 responses and drive tolerogenic responses to grafts. As an example, following hematopoietic stem cell transfer, the presence of iNKT cells is predictive for survival with a reduction in graft versus host disease (GvHD) in patients and preclinical models.

Accordingly, the present disclosure provides a composition comprising iNKT cells. iNKT cell therapy can be autologous, allogeneic or xenogeneic. In some embodiments, the iNKT cells are isolated from a donor (e.g., a donor that is not the subject). In some embodiments, the iNKT cells are isolated from the subject. In some embodiments, the iNKT cells are allogeneic. In some embodiments, the subject is a human and the donor is a human. In some embodiments, the subject and the donor are allogeneic. The term “allogeneic”, as used herein, is a word denoting tissue and/or cells taken from different individuals of the same species, and the tissue and/or cells are genetically dissimilar and immunologically incompatible. iNKT cells, being restricted for the monomorphic CD1d molecule, are substantially devoid of alloreactivity, allowing them to be used off-the-shelf in a donor-unrestricted manner (e.g., without causing graft-versus-host disease (GvHD)).

In some embodiments, the iNKT cells are isolated (e.g., purified or enriched) from peripheral blood mononuclear cells (PBMCs) from apheresis of the donor. In some embodiments, the isolated iNKT cells are expanded ex vivo. In some embodiments, an initial population of the iNKT cells are purified from PBMCs using a suitable method known in the art (e.g., FACS or MACS). In certain embodiments, iNKT cells are isolated from PBMCs by microbead-bound monoclonal antibody to the iNKT invariant TCR.

In certain embodiments of the invention, the iNKT cells are stimulated ex vivo. In some embodiments, the initial population of the iNKT cells are stimulated by α-galactosylceramide (α-GalCer) or any modified glycolipid thereof, e.g., as described by Zhang et al., α-GalCer and iNKT Cell-Based Cancer Immunotherapy: Realizing the Therapeutic Potentials, Front Immunol. 2019 Jun. 6; 10:1126; Schafer et al., iNKT cell stimulation by glycolipid ligands modified from α-galactosylceramide results in differential interleukin-2 secretion profiles, J Immunol May 1, 2019, 202 (1 Supplement) 177.1) for activation and expansion. In some embodiments, the initial population of the iNKT cells are stimulated by α-GalCer while being co-cultured with PBMCs. In some embodiments, the PBMCs are irradiated prior to being co-cultured with iNKT cells. In some embodiments, the PBMCs are pulsed with α-GalCer. In some embodiments, iNKT cells are stimulation with αGalCer-pulsed irradiated PBMCs. In some embodiments, the iNKT cells can go through more than one round of stimulation as described herein.

In certain embodiments, the iNKT cells are expanded ex vivo by culturing with IL-2, IL15, and/or IL-21. For instance, in certain embodiments, iNKT cells are cultured in IL-2 over a period of time where IL-2 is added to the media one or more times. In certain embodiments, the iNKT cells are expanded with IL-15 alone or with IL-21. In certain embodiments, the iNKT cells are expanded ex vivo by culturing the cells with IL-21. In certain embodiments, the iNKT cells are expanded concurrent with stimulation. In certain embodiments, the iNKT cells are stimulated and then expanded. In certain embodiments, the iNKT cells are expanded and then stimulated.

After expansion, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the cells in the composition are iNKT cells.

In certain embodiments of the invention, allogeneic iNKT cells are prepared comprising the steps of i) isolation of iNKT cells from PBMCs by microbead-bound monoclonal antibody to the iNKT TCR, ii) stimulation with the iNKT-specific ligand αGalCer-pulsed irradiated PBMCs and iii) interleukin-2 (IL-2) driven expansion of iNKT cells over several weeks, followed by iv) cell culture harvest, formulation, aseptic filling and cryopreservation.

In some embodiments, the iNKT cells are unmodified (e.g., not genetically modified to express exogenous genes). The present disclosure also contemplates the use of iNKT cells isolated and/or expanded using any suitable known methods in the art, e.g., methods described in

In some embodiments, expanded, unmodified iNKT cells express both Th1 type cytokines (e.g., IFNγ, TNFα, GM-CSF) and Th2 type cytokines (e.g., IL-4, IL-13). In some embodiments, after expansion, the iNKT cells retain their inherent cytotoxic capacity against CD1d-expressing cells. In some embodiments, the iNKT cell therapy composition is AgenT-797 (see, e.g., Yigit et al., 164 AgenT-797, a novel allogenic and ‘off-the shelf’ iNKT cell therapy promotes effective tumor killing.

The iNKT cells described herein may be characterized by reference to certain functional properties. In some embodiments, the iNKT cells described herein may possess one or more of the following properties (e.g. when administered to a subject, such as a subject described herein and/or a subject treated as described herein): capable of treating SARS-CoV-2 (COVID-19); capable of treating moderate to severe SARS-CoV-2; capable of treating acute respiratory distress syndrome (ARDS); capable of treating moderate to severe ARDS; capable of treating moderate to severe ARDS that is secondary to SARS-CoV-2 or influenza; capable of treating patients with SARS-CoV-2 (and/or ARDS) requiring invasive mechanical ventilation (IMV); capable of treating patients with SARS-CoV-2 (and/or ARDS) requiring vein-to-vein extracorporeal membrane oxygenation (VV ECMO); increases survival after 30 days, e.g. for patients requiring IMV (e.g. at least 70% survival) compared to a subject that is not administered the iNKT cells; increases survival after 90 days, e.g. for patients on VV ECMO (e.g. at least 70% survival) compared to a subject that is not administered the iNKT cells; reduces likelihood of oxygenator failure compared to a subject that is not administered the iNKT cells; reduces risk/incidence of contracting/developing concomitant infections (e.g. <40% compared to a subject that is not administered the iNKT cells; reduces risk/incidence of contracting/developing pneumonia (e.g. Ventilator-Associated Pneumonia (VAP)) compared to a subject that is not administered the iNKT cells; reduces risk/occurrence of organ failure of one or more organs compared to a subject that is not administered the iNKT cells; reduces risk/occurrence of renal failure, hepatic failure, hematologic failure, and/or neurological failure compared to a subject that is not administered the iNKT cells; does not induce T cell therapy-related cytokine release syndrome (CRS); induces an anti-inflammatory response; increases an anti-inflammatory response compared to a subject that is not administered the iNKT cells; reduces expression/secretion of pro-inflammatory cytokines (e.g. IL-2, IL-1α/1β, IL-6, etc.) compared to a subject that is not administered the iNKT cells; increases expression/secretion anti-inflammatory cytokines (e.g. IL-1RA etc.) compared to a subject that is not administered the iNKT cells; increases expression/secretion of growth factors (e.g. VEGF-D) an anti-inflammatory response; demonstrates a favorable safety profile (e.g. does not induce adverse events and/or treatment-emergent adverse events); does not induce a dose-limiting toxicity; demonstrates a favorable cell persistence (e.g. of at least 28 days for 1000 million cell dose); demonstrates translocation/distribution from blood to tissue; induces a Transient Donor Specific Allo-Antibody (DSA) Response; and/or reduces DSA response when cells are HLA-class I matched with subject.

In some embodiments, the composition further comprises other components such as cytokines (e.g., IL-2 or IL-15) or cell populations. Briefly, pharmaceutical compositions of the present disclosure may comprise an iNKT cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials. In some embodiments, compositions of the present disclosure are formulated for intravenous administration.

Without further elaboration, it is believed that one skilled in the art can, based on the above description, utilize the present disclosure to its fullest extent. Particular embodiments are, therefore, to be construed as merely illustrative, and not limitative of the remainder of the disclosure in any way whatsoever.

### EXAMPLES

The following examples are provided for illustrative purposes and are not intended to limit the scope of the disclosure.

### Example 1: Invariant Natural Killer T (iNKT) Cell Therapy in Subjects with Moderate Acute Respiratory Distress Syndrome Secondary to Viral Infection

The following example shows that iNKT cell therapy as described herein is effective to improve survival; reduce inflammatory response; reduce occurrence or severity of concomitant infections (e.g., pneumonia); and/or reduced occurrence or severity of organ failure subsequent to ARDS in ARDS patients on invasive mechanical ventilation or Veno-Venous Extracorporeal Membrane Oxygenation (VV ECMO). Further, iNKT cell therapy provides at least the following benefits: (i) demonstrates a favorable safety profile (e.g., no neurotoxicity or grade≥3 TRAE were observed); (ii) demonstrates transient persistence in the periphery consistent with in vivo data describing rapid translocation of iNKT cells from blood into tissues; (iii) opportunity for repeated dosing (while alloantibodies were detected after iNKT cell administration and correlates with degree of HLA matching, the antibody response is transient); and (iv) ability to treat viral diseases and infections (a reduced incidence of Pneumonia was seen in patients treated at the highest dose of iNKT cell therapy).

Invariant natural killer T (iNKT) cells act as master regulators of immune responses, making them an ideal immunotherapy. The present disclosure, in part, is directed to treating viral diseases of the lungs using ex vivo expanded allogeneic iNKT cells for the treatment of a broad spectrum of disease, including viral diseases of the lungs.

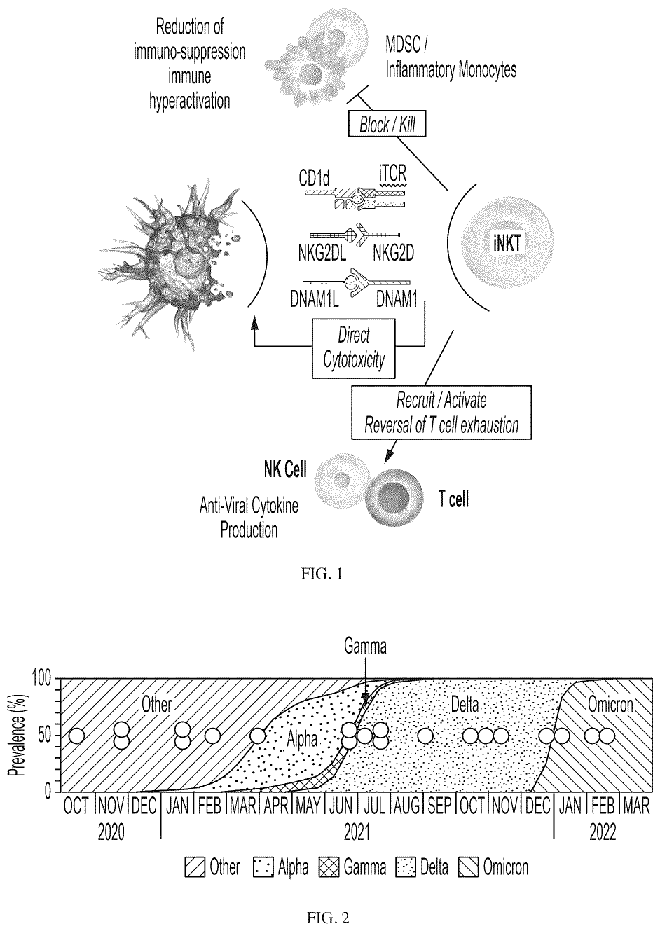

Not wishing to be bound to any particular theory, iNKT cells exert their anti-viral effect via at least the following mechanisms: (i) Direct viral killing: TCR-dependent manner by recognition of CD1d ligands in diseased tissue and activation through the invariant TCR; (ii) Recruitment of host immunity by recruiting host T cells and NK cells: activate NK cells in TCR-independent manner by recognition of stress-signals through activating NK receptors (e.g., NKG2D, DNAM1): (iii) modulation and/or destruction of myeloid suppressor cells and inflammatory monocytes which protects airway epithelium; (iv) recruitment and activation of NK and T cells through cytokine secretion; (v) reversal of T cell exhaustion such as restoring the cytotoxicity capacity, activation and production of partially exhausted T cells; (vi) induce maturation of immature dendritic cells; (vii) dampen pro-inflammatory cytokines; and (viii) cytokine mediated control of bacterial infections, including pneumonia (FIG. 1).

A Phase 1/2 study is ongoing to evaluate the safety and potential efficacy of a cell therapy using unmodified, allogeneic invariant natural killer T (iNKT) cells, in participants with moderate to severe acute respiratory distress syndrome (ARDS) secondary to SARS-CoV-2 or influenza, either with intubation or at high risk to be intubated, as determined using Berlin definition(s). Part 1 employed a standard 3+3 dose escalation design of iNKT cells. All participants received a single infusion of iNKT cells. Participants also received other treatments and supportive care per discretion of the investigator. Once the maximum tolerated dose of iNKT cells has been cleared in Part 1, an Expansion Cohort will be opened. All participants were divided into three cohorts and received a single infusion of iNKT cells at different dosage: Cohort 1 received 100×10\hat{ }6 iNKT cells; Cohort 2 received 300×10\hat{ }6 iNKT cells; and Cohorts 3 received 1000×10\hat{ }6 iNKT cells.

Male and female subjects (aged >18 years) with evidence of SARS-Cov-2-infection with the diagnosis of moderate to severe ARDS secondary to SARS-Cov-2 or influenza per Berlin Definition (2012).

Primary outcome measures include: (i) number of patients with treatment-emergent adverse events; and (ii) number of patients with dose-limiting toxicity (DLT).

Secondary measures include: (i) time to extubation (up to 30 days); (ii) mean daily sequential organ failure assessment score; (iii) change from baseline in C-reactive protein; (iv) decay in quantitative viral burden from upper and lower respiratory tract samples; (v) time from dosing to viral clearance (up to day 30); and (vi) number of participants experiencing viral reactivation and fungal infections.

Summary of participant demographics by dose level cohort is shown in Table 1. Patients' COVID-19 diagnosis date in the context of SARS-CoV-2 strain prevalence in the US is shown in FIG. 2. Patients were treated at three sites in the US in a SARS-CoV-2 variant agnostic manner.

TABLE 1
Patient demographics by dose level cohort

| Variable | Cohort 1 | | Cohort 2 | | Cohort 3 | | Total | |
|---|---|---|---|---|---|---|---|---|
| dose level (cells) | 100 × 10\hat{ }6 | | 300 × 10\hat{ }6 | | 1000 × 10\hat{ }6 | | | |
| Subjects dosed (n) | 3 | | 4 | | 13 | | 20 | |
| Age | | | | | | | | |
| Median (range) | 67 | (66-77) | 71.5 | (64-75) | 62 | (26-75) | 66.5 | (26-77) |
| Median | 67 | | 71.5 | | 62 | | 66.5 | |
| Sex, n (%) | | | | | | | | |
| Male | 2 | (66.7) | 1 | (25.0) | 7 | (53.8) | 10 | (50.0) |
| Female | 1 | (33.3) | 3 | (75.0) | 6 | (46.2) | 10 | (50.0) |
| Patient disposition | | | | | | | | |

| | | | | | | | |
|---|---|---|---|---|---|---|---|
| Early Discontinuation | 10 | 1 | (25.0) | 5 | (38.5) | 6 | (30.0) |
| Death | 0 | 1 | (25.0) | 5 | (38.5) | 6 | (30.0) |

After iNKT cell treatment, adverse effects were evaluated and the representative results are shown in Table 2.

TABLE 2
Adverse Effect

| | Cohort 1 | | Cohort 2 | | Cohort 3 | | Overall | |
|---|---|---|---|---|---|---|---|---|
| | (n = 3) | | (n = 4) | | (n = 13) | | (n = 20) | |
| | n (%) | | n (%) | | n (%) | | n (%) | |
| Any AE | 3 | (100.0) | 4 | (100.0) | 13 | (100.0) | 20 | (100.0) |
| Any AE of grade ≥3 | 3 | (100.0) | 4 | (100.0) | 12 | (92.3) | 19 | (95.0) |
| Any TRAE | 1 | (33.3) 2 | 3 | (75.0) 5 | 1 | (7.7) 1 | 5 | (25.0) |

| | | | | | | |
|---|---|---|---|---|---|---|
| Any TRAE of grade ≥3 | 0 | 1 | (25.0) | 0 | 1 | (5.0) |

| | | | | |
|---|---|---|---|---|
| Any TRAE leading to | 0 | 0 | 0 | 0 |
| discontinuation | | | | |
| Any TRAE leading to | 0 | 0 | 0 | 0 |
| dose interruption | | | | |
| Any TRAE leading to | 0 | 0 | 0 | 0 |
| death | | | | |

Overall, iNKT cell therapy was well tolerated. The majority of observed adverse events (AEs) were consistent with the subjects' underlying diagnosis of severe Covid-19/ARDS requiring invasive mechanical ventilation (IMV). No dose-limiting toxicities were reported. Treatment-Emergent Adverse Events (TEAEs) were observed in all subjects and were consistent with a diagnosis of severe COVID-19/ARDS, including most frequently anemia (n=8), fever (n=7), and acute kidney injury (n=6). TRAE of grade ≥3 was experienced by 1 subject (dyspnea, grade 4).

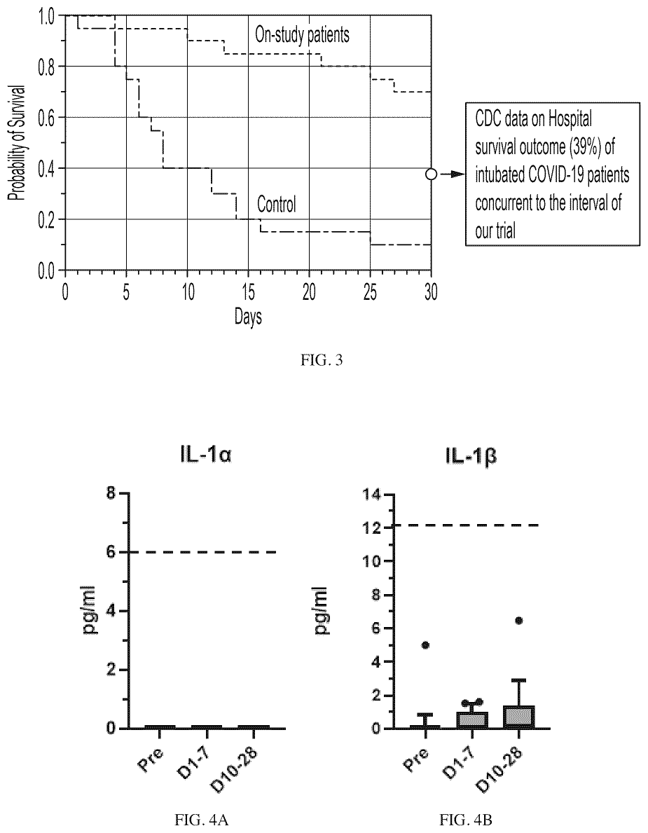

Patient survival was analyzed by Kaplan-Mayer survival curve, which demonstrated that on-study survival of 70% 30 days post treatment as compared to the 10% survival in the control group 30 days after onset of IMV (FIG. 3).

Further, incidence of concomitant infections for each cohort was analyzed and summarized in Table 3.

TABLE 3
Cohort level incidence of concomitant infections.

| | | Cohort 1 | | Cohort 2 | | Cohort 3 | | Overall | |
|---|---|---|---|---|---|---|---|---|---|
| | | (n = 3) | | (n = 4) | | (n = 13) | | (n = 20) | |
| | Grade | n (%) | | n (%) | | n (%) | | n (%) | |
| Infections and | | 3 | (100.0) | 4 | (100.0) | 6 | (46.2) | 13 | (65.0) |
| infestations | | | | | | | | | |
| Pneumonia | 1-4 | 2 | (66.7) | 3 | (75.0) | 2 | (15.4) | 7 | (35.0) |

| | | | | | | | | |
|---|---|---|---|---|---|---|---|---|
| Bacteraemia | 2-3 | 2 | (66.7) | 0 | 1 | (7.7) | 3 | (15.0) |

| | | | | | | | | |
|---|---|---|---|---|---|---|---|---|
| Urinary tract infection | 4 | 0 | 3 | (75.0) | 1 | (7.7) | 3 | (15.0) |
| Fungaemia | 2-3 | 0 | 1 | (25.0) | 1 | (7.7) | 2 | (10.0) |

| | | | | | | | |
|---|---|---|---|---|---|---|---|
| Cytomegalovirus viraemia | 3 | 0 | 0 | 1 | (7.7) | 1 | (5.0) |

| | | | | | | | |
|---|---|---|---|---|---|---|---|
| Lung abscess | 2 | 1 | (33.3) | 0 | 0 | 1 | (5.0) |

| | | | | | | | |
|---|---|---|---|---|---|---|---|
| Pneumonia klebsiella | 3 | 0 | 1 | (25.0) | 0 | 1 | (5.0) |

| | | | | | | | |
|---|---|---|---|---|---|---|---|
| Sepsis | 4 | 1 | (33.3) | 0 | 0 | 1 | (5.0) |

| | | | | | | | |
|---|---|---|---|---|---|---|---|
| Septic shock | 5 | 0 | 0 | 1 | (7.7) | 1 | (5.0) |

| | | | | | | | |
|---|---|---|---|---|---|---|---|
| Upper respiratory | 2 | 1 | (33.3) | 0 | 0 | 1 | (5.0) |
| tract infection | | | | | | | |

Cohort 3 (highest dose level) showed reduced incidence of concomitant infections. An approximate 50% reduction of overall incidence of reported concomitant infections was observed for Cohort 3 compared to the incidence in Cohort 1 and 2 (100% in Cohorts 1 and 2 compared to 46% in Cohort 3). For the incidence of concomitant cases of pneumonia, Cohort 3 showed an over 80% reduction compared to combined numbers of Cohorts 1+2 (Incidence of 15% compared to 71%). By comparison, the published incidence of Ventilator-Associated Pneumonia (VAP) in COVID ARDS ranges from 25%-84%.

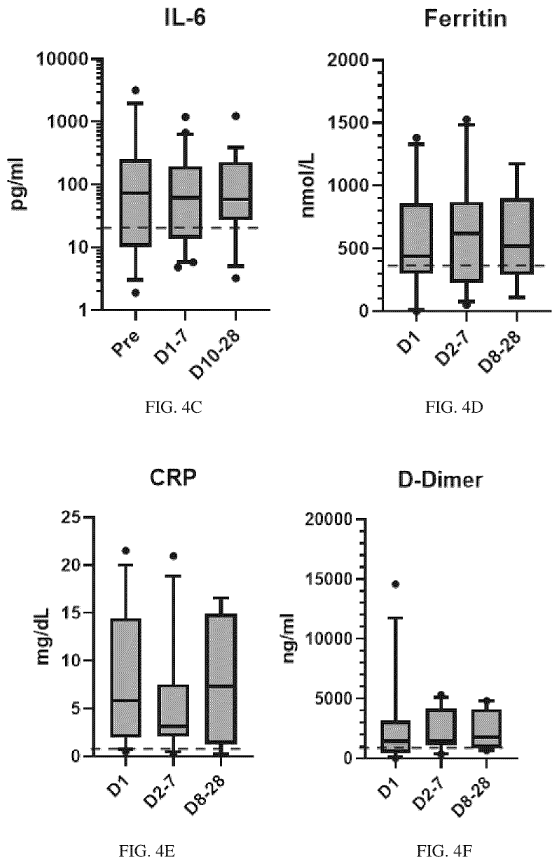

Cytokine release syndrome (CRS) may be a feature of Acute Respiratory Distress Syndrome (ARDS) secondary to viral infection (e.g., influenza or SARS-Cov-2). Therefore, serum levels of key indicators of CRS, e.g., IL-1α, IL-1β, IL-6, Ferritin, C-Reactive Protein (CRP) and D-Dimer, were measured in subjects receiving iNKT cell therapy over 28 days. Cytokine data were binned into pre-infusion sample (Pre, single timepoint), early post-infusion window (from 2 hr post infusion on day 1 to day 7 post-infusion; D1-7) corresponding to measured persistence of iNKT cells in the periphery, and a late post-infusion time window (days 10-28; D10-28). Data were collected for Ferritin, CRP, D-Dimer for day of treatment (D1, single timepoint), early post infusion window (days 2-8; D2-8) and late post-infusion time window (days 8-28; D8-28). Dotted lines indicted upper limit normal level of respective biomarker in healthy people. Among the cytokines tested, IL-1α/β were not detected or within normal range (FIG. 4A-4B); and IL-6, Ferritin, CRP and D-Dimer were elevated in patients' pre-infusion, and levels did not increase post-infusion (FIGS. 4C-4F). The results indicate that treatment with iNKT cells does not induce CRS.

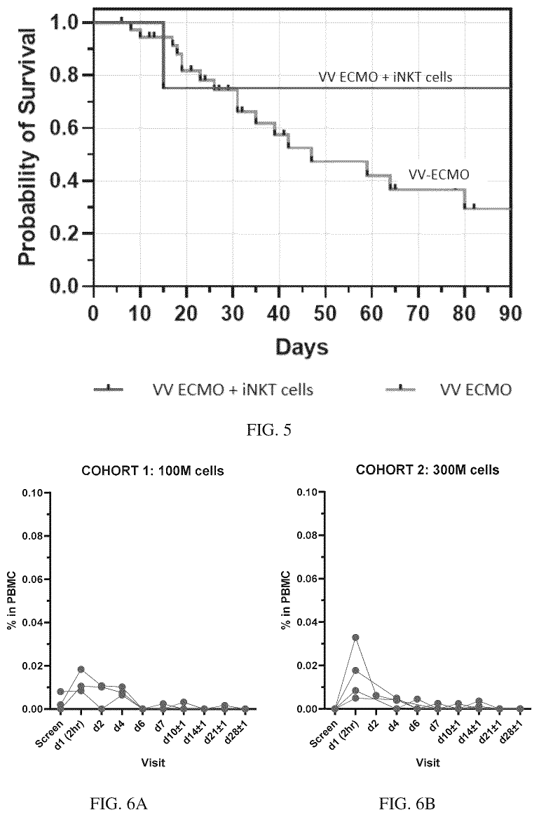

Patients with severe ARDS can be placed on vein-to-vein extracorporeal membrane oxygenation (VV ECMO), which may improve survival of these patients. However, mortality rate is still high in ARDS patients on VV ECMO. In the present study, patients on VV ECMO treated with iNKT cells showed 90-day survival rate of 75% compared to the control cases (FIG. 5). Median survival time of patients treated with iNKT cells (n=4) was 119.5 days compared to 47 days in the control data set (n=36). In ECMO, blood flows through tubing to an artificial lung in the machine that adds oxygen and takes out carbon dioxide, allowing the patient's heart and lungs to rest. In patients treated with iNKT cells, no cell-therapy-associated oxygenator failure due to clogging of filters was observed as is seen with mesenchymal stem cell therapy in ARDS patients on ECMO.

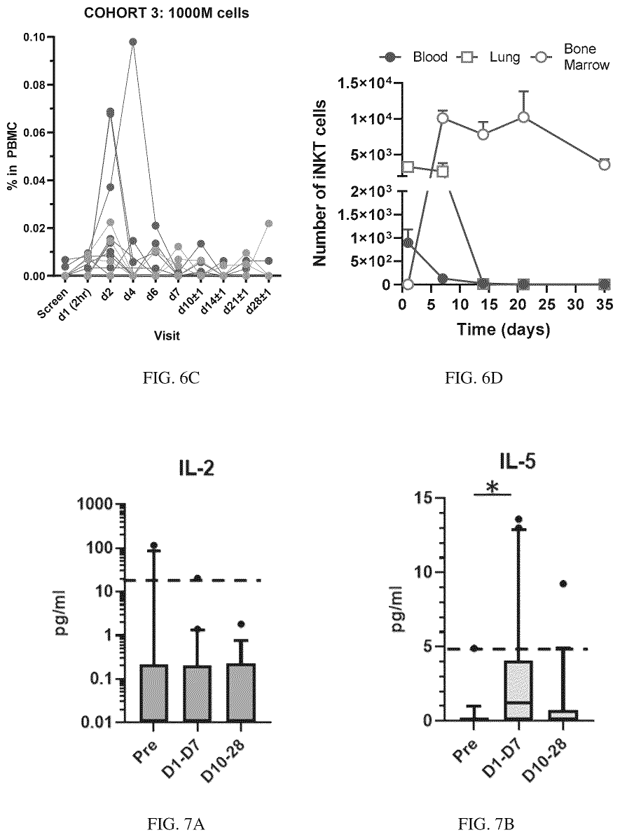

After infusion of iNKT cells, iNKT cell persistence in blood was evaluated. Quantification of infused iNKT cells in patient PBMC was done by digital PCR based on genetic markers unique to donor material. Each line represents data from one patient. Infused iNKT cells were detected up to day 6 post infusion, with likely low-level persistence detectable at the highest dose level for longer, up to the last day of sampling at day 28 (FIGS. 6A-6C). Peak levels of infused iNKT cells demonstrate dose-proportional relationship. Data from patients on ECMO is shown in red. Previous trials of mesenchymal stem cell therapy (MSC) in ECMO patients have reported a high incidence of oxygenator failure due to filter clogging. Detection of infused iNKT cells for prolonged periods post-infusion in ECMO patients is consistent with the lack of filter clogging in ECMO patients received the iNKT cell therapy. Dynamics of tissue distribution of iNKT cells in a murine xenograft model showed rapid translocation of iNKT cells to tissue following intravenous injection (FIG. 6D). The observed transient post-infusion persistence of iNKT cells in patient blood was consistent with the dynamics of blood-to-tissue distribution of iNKT cells in vivo.

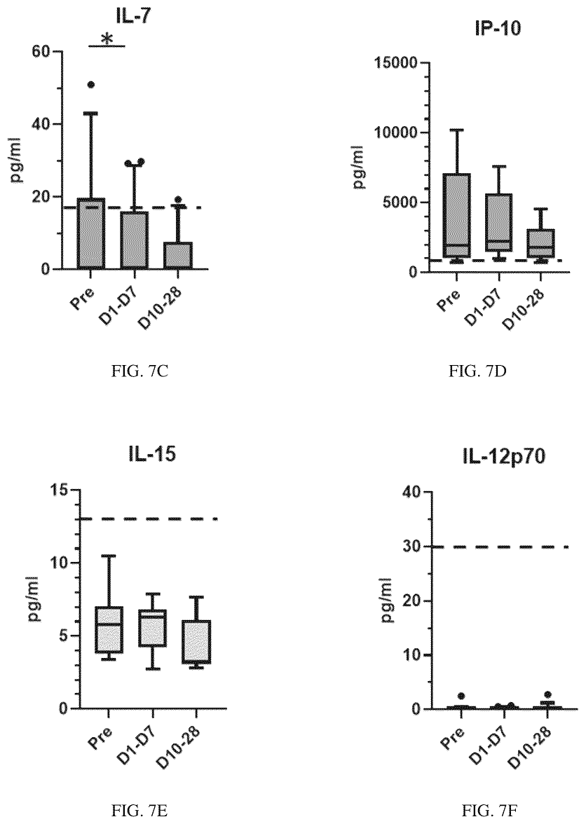

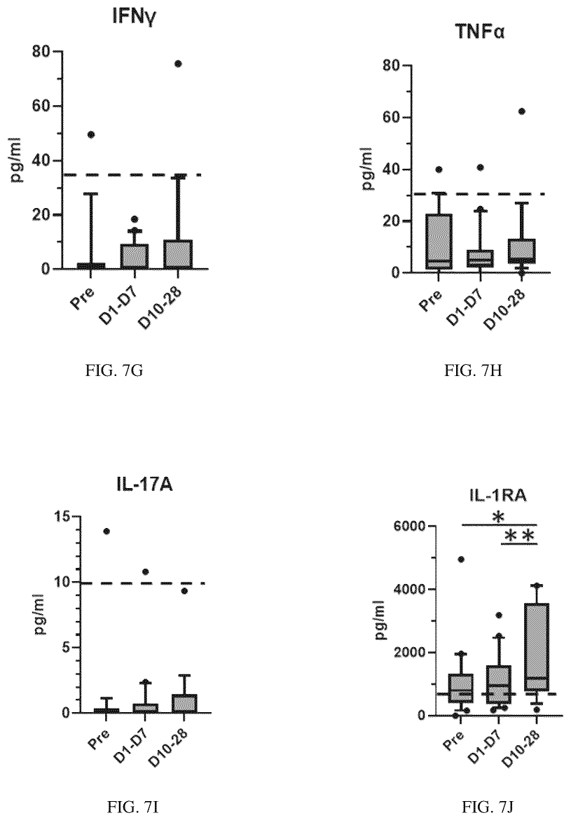

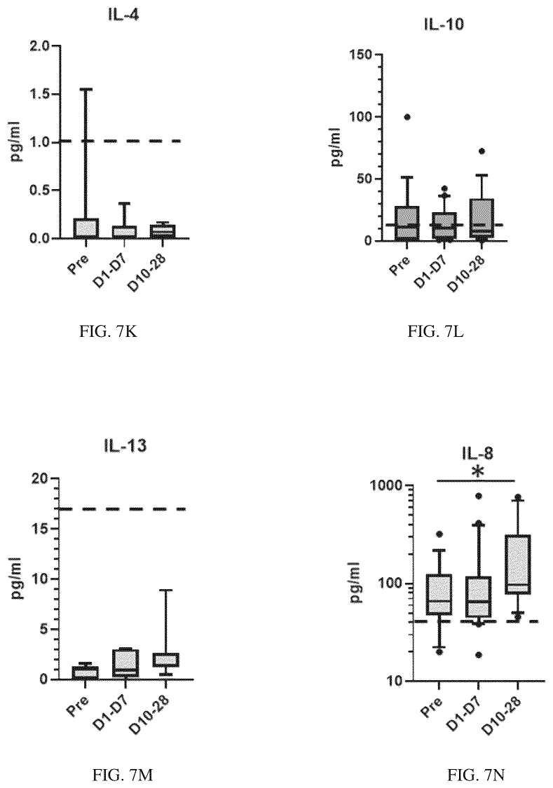

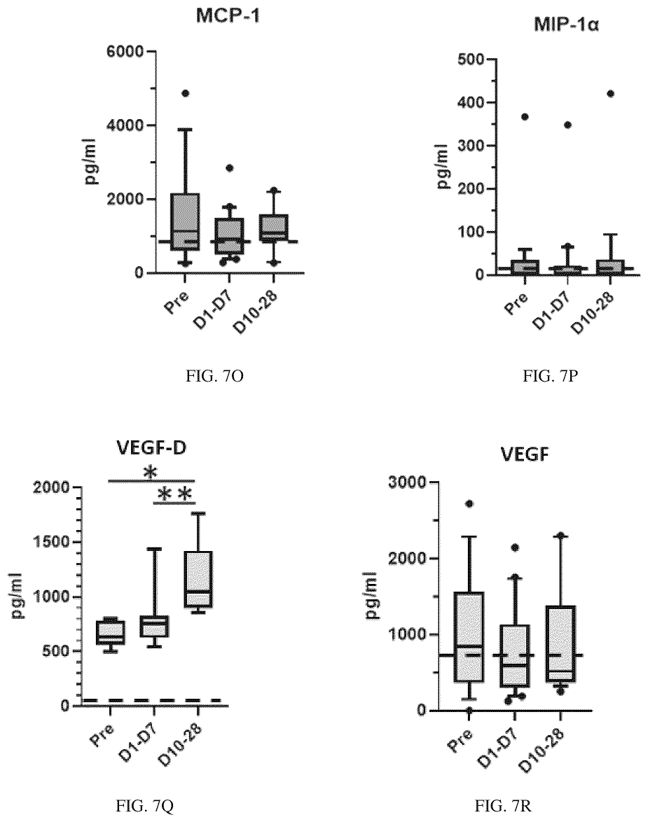

Increased anti-inflammatory response post-infusion of iNKT cell therapy was observed (FIGS. 7A-7R). Cytokines of particular interest are those known to play a crucial role in the pathogenesis of COVID-19 are shown in FIGS. 7A, 7C, 7D, 7G, 7H, 7I, 7J, 7L, 7O and 7P. (data for IL-1 and IL-6 shown in FIGS. 4A-4C)). Of these IL-1RA showed the most significant changes in serum levels post-infusion of iNKT cells, consistent with an increased anti-inflammatory response counteracting IL-1 mediated cytokine release. Levels of pro-inflammatory cytokine IL-7 also reduced significantly. Data for IL-4, IL-15, IP-10 and VEGF-D available only for cohorts 1 and 2. Significant changes post dosing are indicated with asterisks (* p<0.05; **p<0.01; data analyzed by one-way ANOVA). Dotted lines indicted upper limit normal level of respective biomarker in healthy people.

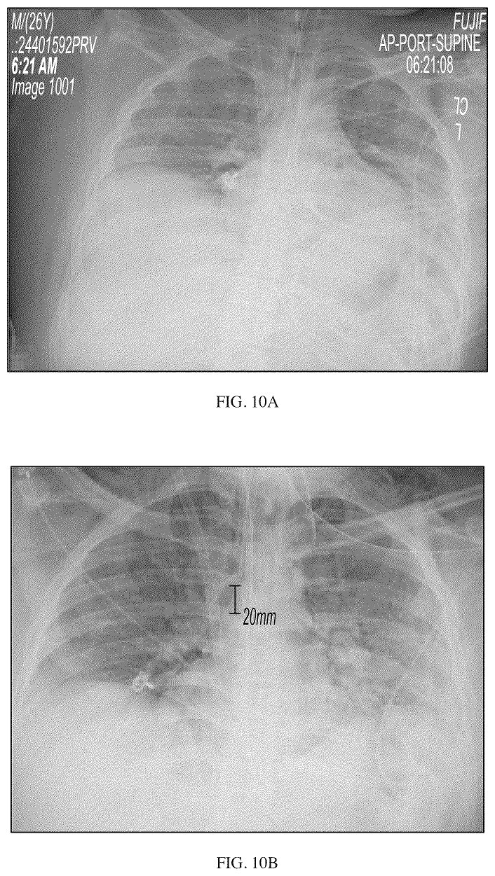

Further, chest X-ray showed improvement of lung function of the patients within 24 hours after iNKT cell infusion compared to chest X-ray before the infusion. (FIGS. 10A-10B). After iNKT cell infusion, the patient had improved lung volumes and stable parenchyma.

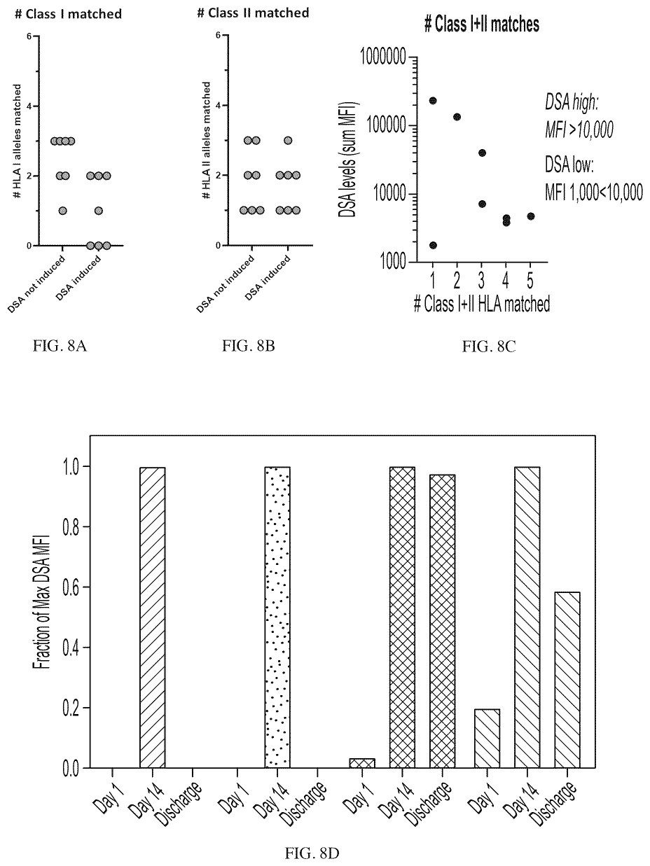

Presence of donor specific allo-antibodies (DSA) was determined on day of dosing and day 14. FIGS. 8A-8B indicate the induction of DSA post iNKT cell infusion. Patients were scored on whether DSA were induced or not induced at Day 14 post infusion. Incidence of DSA development was reduced with increased HLA class I matching but appeared to be unrelated to the degree of HLA-class II matching. Serum levels of DSA post-dosing were reduced with increased degree of HLA matching. DSA levels of combined MFI<1,000 were considered negative and not reported (FIG. 8C). For 4 patients, DSA levels were measured at the time of discharge (day of discharge of patients, from left to right: 60, 28, 21, 28). DSA levels post infusion of iNKT cells peaked at day 14 and appeared to decrease afterwards (data normalized to peak DSA levels for each patient).

Taken together, iNKT cell therapy represents a variant agnostic approach for ARDS patients (e.g., COVID-19 patients with ARDS). Patients treated with iNKT cells demonstrated on-study survival of 70%, compared to 10% survival in the 30-day survival in control-set population and 39% survival in the CDC hospital outcome

iNKT cell therapy showed a favorable safety profile. No neurotoxicity or grade≥3 TRAE were observed. No MTD was determined. iNKT cells also showed transient persistence in the periphery, which was also consistent with in vivo data describing rapid translocation of iNKT cells from blood into tissues. Alloantibody development correlated with degree of HLA matching. However, antibody response appeared transient, suggesting the possibility for redosing. Further, a reduced incidence of pneumonia was observed in patients treated at the highest dose, underscoring the application of iNKT cells in viral diseases and infections more broadly.

### Example 2: Safe Administration of Allogenic iNKT Cell Infusion to Patients with Severe COVID-19 Respiratory Failure Receiving Veno-Venous Extracorporeal Membrane Oxygenation (VV-ECMO) Support

The following example shows that iNKT cell therapy as described herein is effective to improve survival; reduce pneumonia; and/or promote an anti-inflammatory response in ARDS patients on Veno-Venous Extracorporeal Membrane Oxygenation (VV ECMO).

Despite trends toward improved mortality in severe COVID-19 respiratory failure, largely due to early corticosteroid and anti-viral therapy, death rates have remained high (47.9% to 84.4%). Addition of Veno-Venous Extracorporeal Membrane Oxygenation (VV-ECMO) support has improved survival in a select group of COVID-19 patients, but overall mortality at 90 days is still 47%. Complications of VV-ECMO therapy are myriad, including bleeding, oxygenator failure and hospital acquired infections. There is a need for multi-modal therapies in patients with severe COVID-19 respiratory failure that can augment current interventions. Ideally, such therapies would modulate the initial hyperinflammatory response, but also fortify the innate immune system against other hospital acquired threats. Invariant natural killer T (iNKT) cells comprise ˜0.8% of leukocytes in healthy human hosts and naturally home to damaged organs, including lungs, where they dampen proinflammatory cytokines and protect epithelial tissues. Low circulating levels of iNKT cells may be a marker of COVID-19 ARDS mortality (Kreutmair et al., Distinct immunological signatures discriminate severe COVID-19 from non-SARS-CoV-2-driven critical pneumonia, Immunity. 2021 Jul. 13; 54(7):1578-1593.e5). In severe cases of COVID-19, circulating iNKT cells have been shown to be activated by IL-18 (Jouan et al., (2020) Phenotypical and functional alteration of unconventional T cells in severe COVID-19 patients. J Exp Med 217(12)), which is a cytokine associated with unconventional T cell activation during viral infections (Tsai et al., Type I IFNs and IL-18 regulate the antiviral response of primary human 76 T cells against dendritic cells infected with dengue virus. J. Immunol. 2015; 194(8):3890-3900; Tyznik et al., Distinct requirements for activation of NKT and NK cells during viral infection. J. Immunol. 2014; 192(8):3676-3685; Treiner E et al (2003) Selection of evolutionarily conserved mucosal-associated invariant T cells by MR1. Nature 422(6928):164-9. 10.1038/nature01433). Furthermore, there is an observed decrease in circulating iNKT cells and in iNKT cell production of IFNγ. Of the remaining iNKTs in circulation, levels of those expressing PD-1 and CD69 were increased while high PD-1 expression persisted on iNKT cells from patients in intensive care units at day 15 (Jouan et al., (2020) Phenotypical and functional alteration of unconventional T cells in severe COVID-19 patients. J Exp Med 217(12); Orumaa et al., The role of unconventional T cells in COVID-19, Ir J Med Sci. 2022; 191(2): 519-528; Liu et al., Analysis of the Long-Term Impact on Cellular Immunity in COVID-19-Recovered Individuals Reveals a Profound NKT Cell Impairment, ASM Journals mBio, Vol. 12, No. 2).

In the present study, four male patients, average age 50 years with severe COVID-19 pneumonia met established criteria for VV-ECMO cannulation. All four received dexamethasone and remdesivir and were treated with a one-time, 1000×10⁶ iNKT cell infusion. The primary end point was safety and tolerability and multiple secondary and exploratory end points were identified, including all-cause mortality at 30 days.

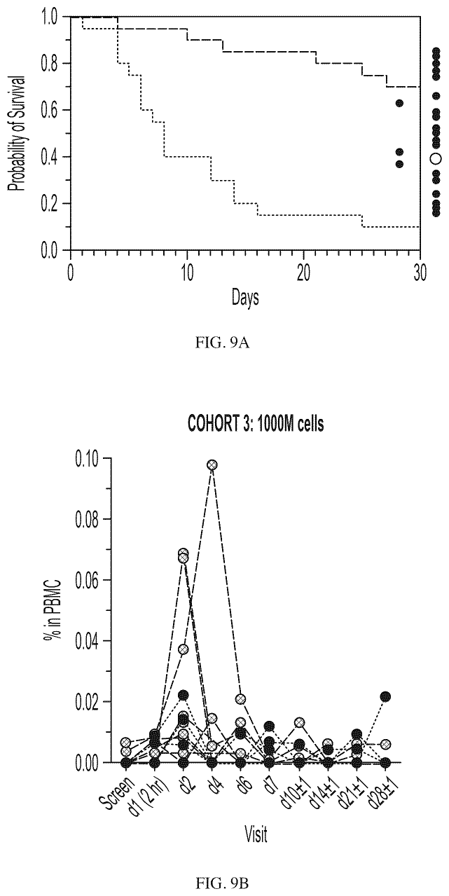

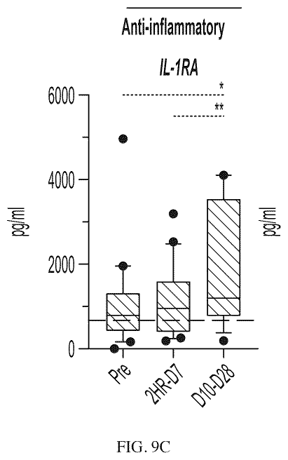

No significant adverse infusion related events, including oxygenator failure, were reported. Survival was 100% at 14 days and 75% at 30 and 90 days with an average ECMO run time of 133.5 days (FIG. 9A). One patient died at day 15 of fulminant liver failure unrelated to the study drug. Persistence of circulating iNKT cells in the VV-ECMO cohort was comparable to study patients not on ECMO (FIG. 9B) and decreased rates of pneumonia were observed in comparison to a single center VV-ECMO 2021-2022 cohort (n=38). Of note, significantly increased levels of the anti-inflammatory cytokine IL1-RA (FIG. 9C) and growth factor VEGF-D were also observed.

iNKT cell therapy was well tolerated in patients with severe COVID-19 ARDS receiving VV-ECMO. Overall survival at 30 days was 75%. Exploratory data such as hospital acquired infection rates and protective cytokine levels in treated patients showed statistically relevant and potentially important trends. These early observations support the efficacy of iNKT cell-based immunotherapies in the treatment of ARDS (e.g., ARDS associated with COVID-19).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms. Definition of terms are disclosed throughout the specification, including but not limited to the “Definition” section.

The section heads are not meant to be interpreted to limit the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method for treating a subject having acute respiratory distress syndrome (ARDS), the method comprising administering to the subject a composition comprising invariant natural killer T (iNKT) cells.

2. A method for reducing or preventing organ damage in a subject having acute respiratory distress syndrome (ARDS) or at risk for organ failure, the method comprising administering to the subject a composition comprising invariant natural killer T (iNKT) cells.

3. A method for inducing an anti-inflammatory response in a subject having acute respiratory distress syndrome (ARDS), the method comprising administering to the subject a composition comprising invariant natural killer T (iNKT) cells.

4. A method of reducing or preventing concomitant infections in a subject having acute respiratory distress syndrome (ARDS), the method comprising administering to the subject a composition comprising invariant natural killer T (iNKT) cells.

5. A method of reducing or preventing concomitant infections in a subject receiving invasive mechanical ventilation or veno-venous extracorporeal membrane oxygenation (VV ECMO), the method comprising administering to the subject a composition comprising invariant natural killer T (iNKT) cells.

6. A method of reducing or preventing a hospital acquired infection in a subject at risk thereof, the method comprising administering to the subject a composition comprising invariant natural killer T (iNKT cells).

7. The method of claim 5, wherein the subject has acute respiratory distress syndrome (ARDS).

8. The method of claim 1, wherein the iNKT cells are unmodified.

9. The method of claim 1, wherein the iNKT cells are derived from a donor that is not the subject.

10. The method of claim 1, wherein the iNKT cells are allogeneic.

11. The method of claim 1, wherein the iNKT cells are isolated from peripheral blood mononuclear cells and are expanded ex vivo.

12. The method of claim 1, wherein the donor is a human.

13. The method of claim 1, wherein at least 90% of the cells in the composition are iNKT cells.

14. The method of claim 1, wherein at least 95% of the cells in the composition are iNKT cells.

15. The method of claim 1, wherein the ARDS is associated with a viral infection.

16. The method of claim 15, wherein the viral infection is caused by coronavirus, influenza virus, rhinovirus, parainfluenza virus, adenovirus, respiratory syncytial virus (RSV), or human metapneumovirus.

17. The method of claim 16, wherein the coronavirus is severe acute respiratory syndrome coronavirus-2 (SARS-CoV-2), severe acute respiratory syndrome (SARS) coronavirus (SARS-CoV-1), or Middle East Respiratory Syndrome Coronavirus (MERS-CoV).

18. The method of claim 16, wherein the influenza virus is H1N1 influenza, H5N1 influenza, or H7N9 influenza.

19. The method of claim 1, wherein the subject does not receive mechanical ventilation.

20. The method of claim 1, wherein the subject receives mechanical ventilation.

21. The method of claim 20, wherein the subject is on mechanical ventilation while being administered the composition.

22. The method of claim 1, wherein the subject is refractory to mechanical ventilation.

23. The method of claim 1, wherein the subject receives extracorporeal membrane oxygenation (ECMO).

24. The method of claim 23, wherein the extracorporeal membrane oxygenation is veno-venous extracorporeal membrane oxygenation (VV ECMO).

25. The method of claim 24, wherein there is no oxygenator failure due to clogging.

26. The method of claim 1, wherein the administration of the composition does not induce cytokine release syndrome.

27. The method of claim 1, wherein the administration of the composition improves survival of the subject relative to a subject that is not administered the composition.

28. The method of claim 1, wherein the administration of the composition induces an anti-inflammatory response in the subject as measured by one or more cytokines, wherein the one or more cytokines comprises: IL-1 α/β, IL-6, ferritin, C reactive protein (CRP), IL-2, IL-5, IL-7, IP-10, IL-15, IL-12p70, IFNγ, TFNα, IL-17A, IL-IRA, IL-4, IL-10, IL-13, IL-8, MCP-1, MIP-1α, VEGF, or VEGF-D.

29. The method of claim 1, wherein the administration of the composition reduces the occurrence of concomitant infections relative to a subject that is not administered the composition.

30. The method of claim 4, wherein the concomitant infections are hospital acquired infections.

31. The method of claim 6, wherein the hospital acquired infections comprises Klebsiella aerogenes, catheter-related bloodstream infection due to Candida albicans, ventilator-associated pneumonia (VAP) due to multidrug-resistant Pseudomonas aeruginosa (MDRP).

32. The method of claim 1, wherein the administration of the composition reduces the occurrence of one or more organ failures relative to a subject that is not administered the composition.

33. The method of claim 1, wherein the organ failure comprises renal failure, hepatic failure, hematologic failure, and/or neurological failure.

34. The method of claim 1, wherein the organ failure is renal failure.

35. The method of claim 1, wherein the subject is administered 80×10⁶ to 2000×10⁶ iNKT cells.

36. The method of claim 1, wherein the subject is administered 100×10⁶ iNKT cells.

37. The method of claim 1, wherein the subject is administered 300×10⁶ iNKT cells.

38. The method of claim 1, wherein the subject is administered 100×10⁶ iNKT cells.

39. The method of claim 1, wherein the administration is via intravenous injection or intravenous infusion.

40. The method of claim 1, wherein the subject is administered the composition once.

41. The method of claim 1, wherein the subject can be repeatedly dosed one or more times after the initial dosing.

42. The method of claim 1, wherein the subject is also administered dexamethasone and/or remdesivir.

43. The method of claim 1, wherein the administration results in improved lung function of the subject compared to the lung function of the subject prior to the administration.

44. The method of claim 1, wherein the administration results in increased lung volume of the subject compared to the lung volume of the subject prior to the administration.

45. The method of claim 1, wherein the administration results in increased lung parenchyma stability of the subject compared to the stability of lung parenchyma of the subject prior to the administration.

46. A method for reducing inflammation in a subject in need thereof, the method comprising administering to the subject a composition comprising invariant natural killer T (iNKT) cells.

47. A method for reducing secondary infection in a subject in need thereof, the method comprising administering to the subject a composition comprising invariant natural killer T (iNKT) cells.
